(19) United States
(12) Patent Application Publication
(10) Pub. No.: US 2003/0125771 A1
(43) Pub. Date: Jul. 3, 2003

(54) Multiphasic defibrillator utilizing controlled energy pulses

(75) Inventor: Michael C. Garrett, Stokie, IL (US)

Correspondence Address: TREXLER, BUSHNELL, GIANGIORGI,, BLACKSTONE & MARR, LTD., 105 WEST ADAMS STREET, SUITE 3600, CHICAGO, IL, 60603, US

(73) Assignee: Medical Research Laboratories, Inc.

(21) Appl. No.: 10/209,772

(22) Filed: Aug. 1, 2002

Related U.S. Application Data
(60) Provisional application No. 60/309,294, filed on Aug. 1, 2001.

(51) Int. Cl. A61N 1/39

(52) U.S. Cl. 607/5

(57) ABSTRACT

An expert system-controlled defibrillator for delivering precise energy doses to a patient who's heart is in fibrillation. An energy source connects to the patient's chest (during emergency resuscitation) or directly to the heart (during open-heart surgery) and discharges energy in one or more pulses. The apparatus measures a patient-dependent parameter or parameters, and determines, from an expert based system, the waveform morphology and the precise amount of energy to deliver.

### PRIORITY

This application claims the benefit of U.S. Provisional Application Serial No. 60/309,294, filed on Aug. 1, 2001.

### BACKGROUND OF THE INVENTION

This invention relates to the use of defibrillators to deliver energy to the heart for emergency resuscitation of a patient whose heart has gone into fibrillation. The method of delivering energy to the chest of such a patient is well established. An energy storing device, usually one or more capacitors, is coupled to two electrodes (usually called paddles or pads). The paddles are placed in contact with the chest of the patient (in the case of external defibrillation), or directly to the heart of the patient (in the case of internal defibrillation during open-heart surgery), to apply energy to the heart of the patient. The energy momentarily stops the heart so that fibrillation also stops. When the voltage gradient across the heart decays, the heart will begin contracting normally if the defibrillation event was successful. If a defibrillation pulse is applied to a heart in fibrillation within approximately two minutes of the onset of fibrillation, there is a good chance the heart will begin to contract normally.

The graph of the current or voltage of the energy versus time shows the waveform of the energy delivered. The waveform of the energy delivered is characterized by shape, polarity, duration, and the number of phases. The shape includes the amplitude (voltage or current), the width (time), and the tilt (rate of decay). An exemplary waveform is illustrated in FIG. 2.

Monophasic waveforms were initially used in defibrillation. The use of the application of energy in a biphasic waveform, using lower voltages and lower total energy than with a monophasic waveform, is well established.

Prior art defibrillators measured charge delivered or time of delivery of energy. An improvement of this art was to utilize a patient-dependent parameter to determine the shape of the waveform. Some prior art defibrillators deliver a test pulse to the patient to determine the patient's impedance, which is then used to determine the shape of the waveform by accumulating charge or calculating the required time to deliver the selected energy. By shaping the waveform in this way, the defibrillator must know the exact capacitance of the energy storage device to deliver a precise amount of energy. The maker of the defibrillator accordingly must purchase expensive components in which the capacitance is known to a very high degree, or must utilize a calibration unit within the defibrillator, which adds to the cost and weight of the unit. Additionally, capacitors degrade with use, requiring either the replacement of the capacitor in the device or frequent calibration of the device.

The present invention involves delivery of energy to the patient with a energy protocol and waveform shape determined by an expert system. It is an object of using an expert system to maximize the effectiveness of the defibrillation pulse based on various physical parameters of the patient as well as the patient's ECG morphology or cardiac electrical activity. The expert system used to determine the pulse shape can include knowledge gained in past episodes of defibrillation by using embedded algorithms to determine shock efficacy. The expert system can also be programmed using a rule-based look up table stored in memory using known or proven rules of defibrillation based on current state of the art as described in the preferred embodiment. The expert system can use one or many of the known algorithmic or other approaches known in the art such as look-up tables, neural networks, fuzzy-logic based systems, genetic algorithms and adaptive performance surface searching. The previous list is not all-inclusive and may be added to as technology progresses. The main feature of this invention is the use of an expert-based system.

It is a further object of this invention to deliver energy to the patient on a per pulse basis as determined by the expert system. In the preferred embodiment, the unit measures a patient dependent parameter and uses a table generated from a rule-based expert system to determine the amount of energy to deliver on a per pulse basis. By measuring in real time the energy being delivered to the patient, the unit can compensate for small differences in the capacitor bank value to deliver an accurate amount of energy. Since the characteristics of the pulse are determined by energy, the capacitance of the energy storage unit need not be known with any great degree of certainty and less expensive components, in which the capacitors are not required to have a tight tolerance, so that the actual capacitance may vary from the nominal value, saving on component costs. Additionally, the characteristics of the delivered waveform can be predicted more accurately by the method of the present invention.

By using a rule-based mechanism to choose waveform parameters, the defibrillator waveform can be chosen to maximize effectiveness based on set of patient parameters. This rule-based or expert system can be pre-programmed or programmed at the time of pulse delivery to deliver an appropriate energy and waveshape based on current defibrillation science. Since the rules are stored in memory in the unit, a user or the manufacturer can change the rules used by the expert system as medical studies indicate.

It is a further object of the invention is to provide a precise energy dose to a patient in a monophasic or multiphasic defibrillation waveform by delivering controlled energy pulses, with the energy of each pulse retrieved from a table in memory, using a patient-dependent parameter-derived index.

It is a further object of the invention to provide a defibrillator in which the rules can be changed upon further medical study, so that the device is adaptable to advances in medical research.

It is a further object of the invention to provide a defibrillating apparatus in which the user or the manufacturer can select and edit the values in the tables in memory by modifying the rule base or expert system.

It is a further object of the invention to provide a defibrillator using a large energy storage device, in order to decrease the tilt of the waveform allowing higher terminating currents. By maximizing the terminating current, or tilt, less voltage and current can be used to achieve effective defibrillation. Higher terminating current can also decrease post-shock arrhythmias necessitating further defibrillation events. In the preferred embodiment, a 500 microfarad electrolytic capacitor is used as the energy storage element. Having a capacitor above 300 microfarad allows tilt to be optimized for single phase or multiphasic defibrillation pulses. The tilt is defined as the starting voltage Vₛ minus the ending voltage Vₑ divided by the starting voltage Vₛ (multiply by 100 to get percent tilt).

tilt=(Vₛ−Vₑ)÷Vₛ

### SUMMARY OF THE INVENTION

The present invention delivers a truncated exponential pulse waveform to the patient, of one or more polarities, using a single capacitor as the energy storage device. The energy of the pulses is dependent on the desired total energy, a patient-dependent parameter or parameters, and pulse energies retrieved from a look-up table. In the preferred embodiment, the tilt of the waveform is kept low by using a large storage capacitor. The large capacitor allows the pulse length to be extended to accommodate patients with high impedance, and to prevent re-fibrillation or other complications, by maintaining a high terminating current.

The desired total energy is based on a device-defined or user-defined energy index. The patient-dependent parameter of the preferred embodiment is patient resistance. The look-up table defines how much energy to deliver on each pulse. The table is created by a rule-based generator, using information defined prior to the creation of the table, which is then stored in memory. The user can edit the table, or the apparatus can be programmed to modify table entries based on effectiveness as recorded in past history.

The apparatus 10, by measuring the patient's ECG via the electrode 16, detects that the heart has resumed normal electrical activity, and has potentially begun pumping blood again. The apparatus 10 can be programmed to record the success or failure of a delivered energy pulse, along with the characteristics of that pulse and measured or physical parameters of the patient. The patient's parameters can include weight, pulse, percentage of body fat, ECG or other physiological measurements, or any other parameters that medical studies indicate are relevant to re-fibrillation. The apparatus contains an expert system, which uses one or more of the following: look-up table, neural network, fuzzy-logic based system, genetic algorithm, adaptive performance measures, or error surface searching. The expert system can analyze past data and can adjust energies delivered and or the characteristics of the delivered energy pulses based on that data.

In the preferred embodiment, the apparatus interpolates energy values if required.

### BRIEF DESCRIPTION OF THE DRAWINGS

FIG. 1 is a diagram of the apparatus of the preferred embodiment.

FIG. 2 is a voltage versus time graph illustrating an exemplary biphasic waveform.

FIG. 3 is an exemplary rule-based diagram showing the plot of total energy, patient resistance, and energy ratio.

FIG. 4 a flowchart showing the pulse delivery sequence for a biphasic defibrillator.

FIG. 5 is a flowchart of pulse delivery for a single pulse in the preferred embodiment.

FIG. 6 is an exemplary energy table as implemented in the preferred embodiment of the present invention.

### DESCRIPTION OF THE PREFERRED EMBODIMENT

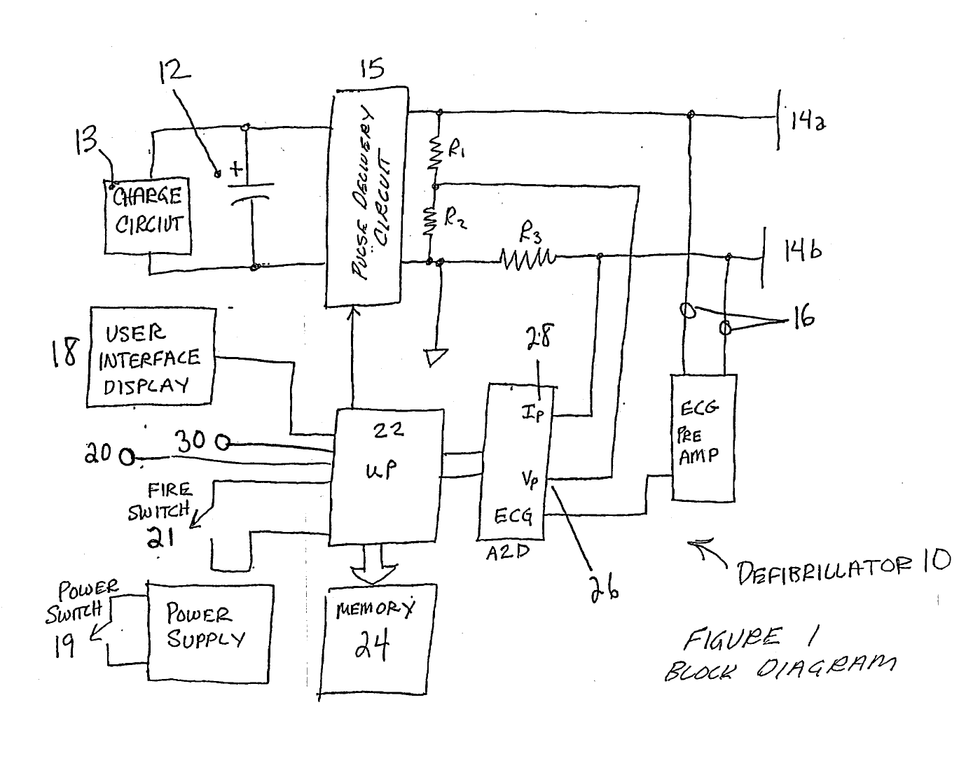

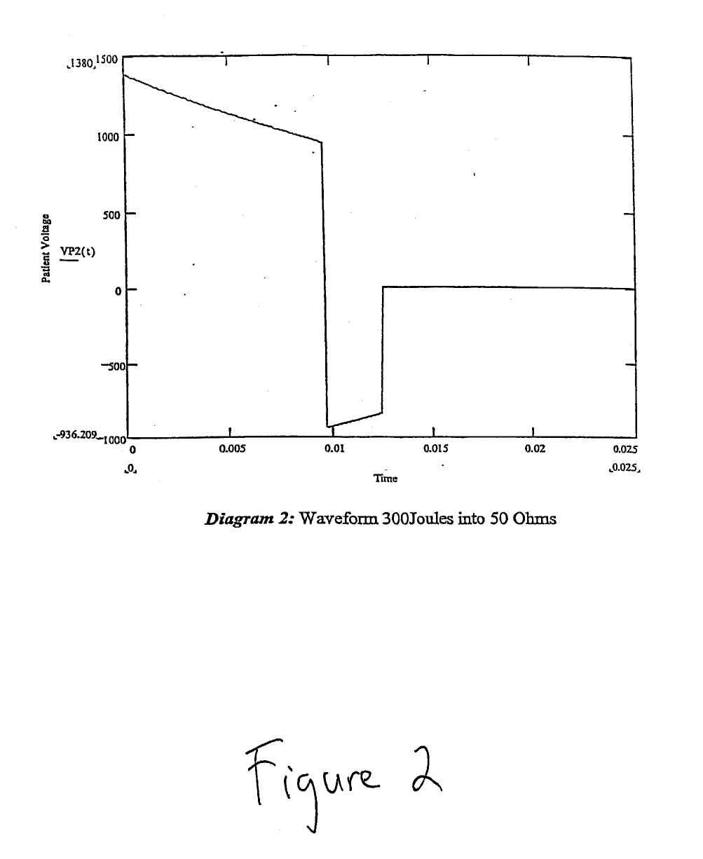

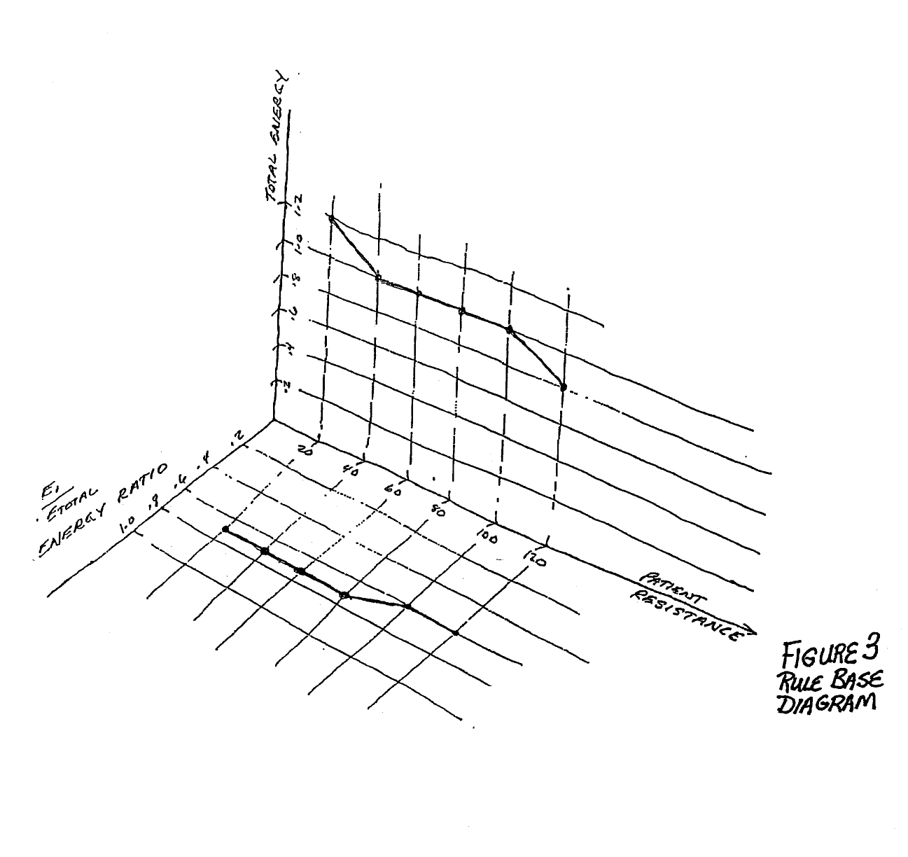

The apparatus 10 is shown in FIG. 1. The apparatus consists of an energy storage capacitor 12, a charger 13, electrodes or paddles 14a and 14b, a pulse delivery circuit 15, electrodes 16 to determine the state of the patient's heart, a user interface display 18, a power switch 19, a charge button 20, a fire switch 21, a microprocessor containing an expert system 22, a memory 24, voltage sampling means 26, current sampling means 28, and a target energy selection control 30.

In a manual embodiment, the user interacts with the apparatus 10. The user turns on the unit by the power switch 19. The user assesses the patient's condition by connecting the ECG electrodes 16 to the patient's chest. If the apparatus 10 detects a shockable heart rhythm, i.e. that a shock is required, the user selects a target energy based on a predetermined protocol. That protocol is based on the American Heart Association/Advanced Cardiac Life Support Guidelines. The user places the paddles or disposable pads 14a and 14b of the apparatus 10 on the patient's bare chest, charges the apparatus 10 by pressing the charge button 20, causing the charging means 13 to charge the capacitor 12, and, when prompted by the apparatus 10, depresses the fire button 21 to deliver the energy. In the preferred embodiment, the target energy selection control 30 has preselected target energy levels of 2.0, 3.0, 4.0, 5.0, 6.0, 7.0, 8.0, 9.0, 10.0, 20.0, 30.0, 50.0, 70.0, 100.0, 150.0, 200.0, 300.0, and 360.0 joules, the actual values dependant on configuration of the apparatus 10 and current medical studies.

In an automatic embodiment, the apparatus 10 chooses the energy to be delivered based on a user-defined energy protocol that can be programmed into the memory 24 at the time of purchase, or modified later by the user, or a value determined by the expert system 22. The user places the electrodes 14a and 14b on the patient's bare chest and depresses the power switch 19. The apparatus 10 analyzes the patient's ECG waveform via electrode 16 and determines whether a shock is required. If required, the apparatus 10 charges up and prompts the user to depress the fire button 21. The apparatus 10 can also deliver the energy without user interaction in a fully automatic mode.

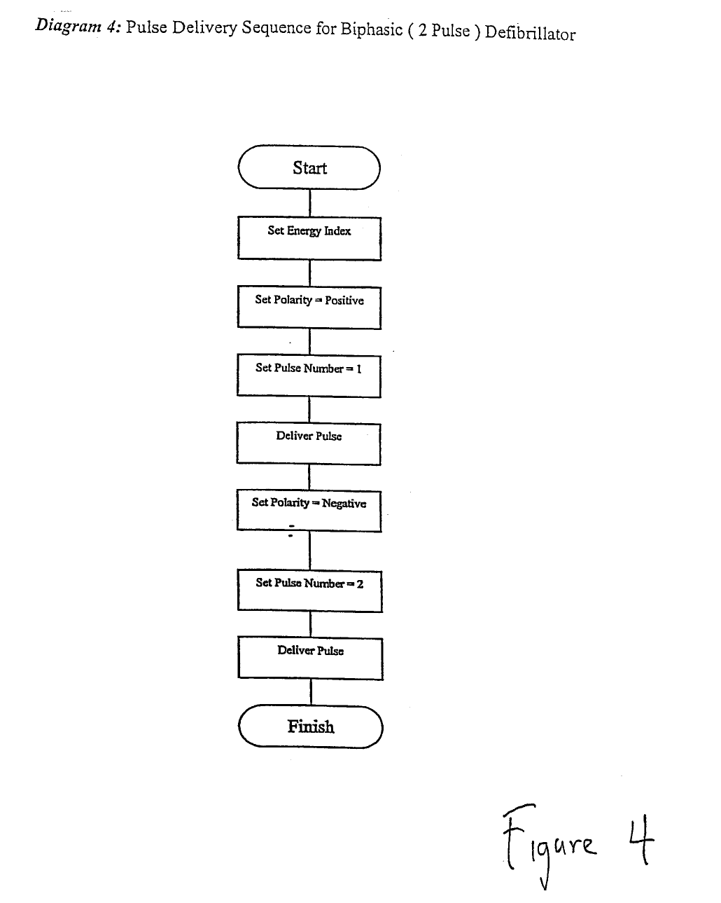

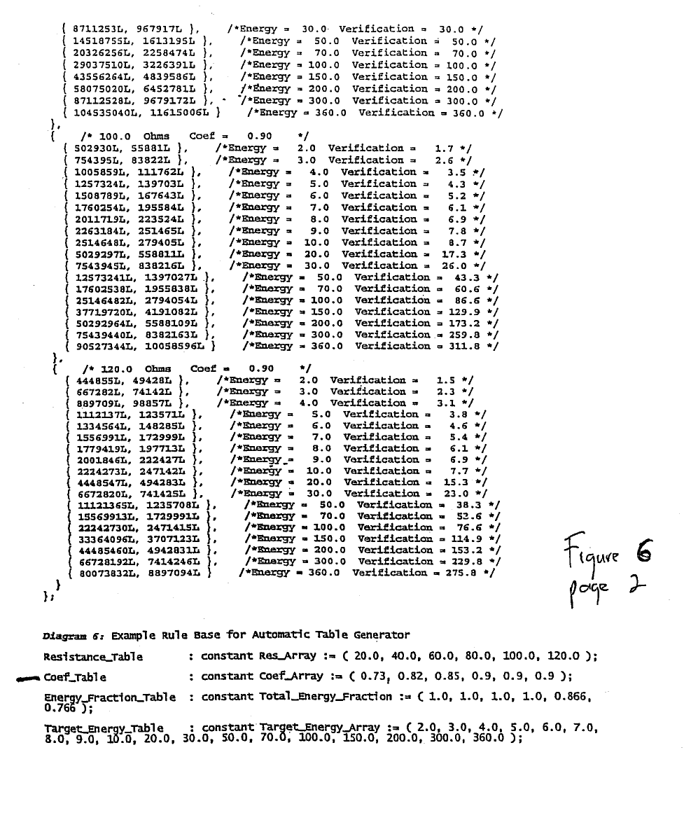

In the preferred embodiment, a rule base is drawn up based on clinical data. The values from the rule base are entered into an expert-system program and an include file is generated containing a table used during operation. An example of a rule base is illustrated in FIG. 3 and a sample energy table is shown in FIG. 6. This table is then compiled into the code and stored in memory 24 for use. In other embodiments the expert system can be contained in the apparatus 10 itself and interacted with by the manufacturer or the user via the front panel, a connected PC or other computer, or remotely.

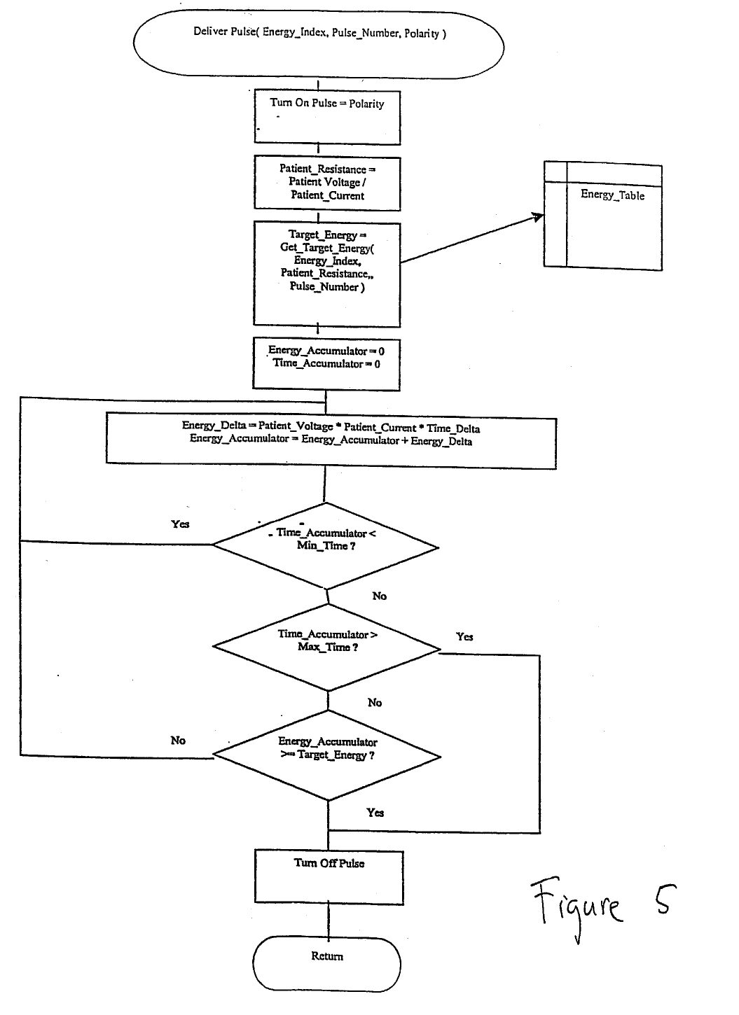

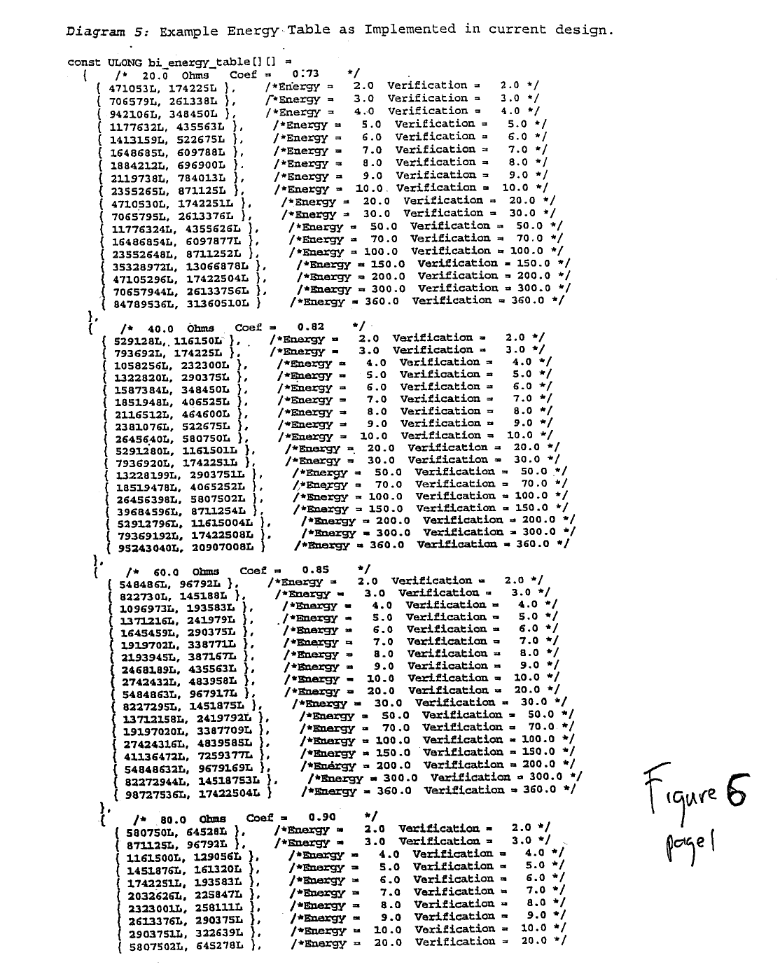

The logic of the application of a biphasic application is shown in FIG. 4. The apparatus 10 begins with a first pulse, with a voltage (V) sufficient to discharge the target energy in 12 mSec into a 50 ohm load. These initial values can be changed as medical studies indicate. The apparatus 10 determines the required starting voltage using the standard equation for energy and solving for Vₛ, the starting voltage:

$$V ( E ) := \frac{- 1}{( C \cdot exp ( \frac{- 3}{125 \cdot Rp \cdot C} ) - C )} \cdot {[ - 2 \cdot C \cdot ( exp ( \frac{- 3}{125 \cdot Rp \cdot C} ) - 1 ) \cdot E ]}^{2}$$

At the start of the first pulse, the apparatus 10 determines the resistance of the patient. The voltage and current (I) are determined continually by sampling the waveform. An exemplary biphasic waveform is shown in FIG. 3. At approximately 400 microsecond into the first pulse, the apparatus 10 takes the voltage and current readings and divides to determine resistance, using the standard equation for calculation of resistance, which equals voltage divided by current:

R=V/i

The apparatus 10 then looks to the rule-based table, illustrated in FIG. 6 and stored in memory 24, to determine how much energy to deliver on this first pulse. The apparatus 10 continues to discharge while integrating the sampled values until the desired energy value has been reached, or until a maximum time is reached in the case of a very highly resistive patient or open load. If a maximum time is reached, the microprocessor 22 signals the pulse delivery circuit 15 to terminate the current.

The apparatus 10 uses the voltage and current of the discharge and integrates over time to determine energy delivered. Voltage readings and current readings are taken approximately every 400 microsecond and multiplied by time to determine energy, using the standard equation:

ΣE=ViΔt

When the apparatus 10 has delivered the desired energy for the first pulse, it truncates the waveform by shutting off current flow, using the pulse delivery circuit 15. The apparatus 10 then waits a predetermined amount of time and starts the delivery of the second pulse.

The apparatus 10 then begins to deliver the second pulse, of opposite polarity, using the same logic as described for the first pulse: turning on the output, calculating the patient resistance by measurement of voltage and current, determining from the rule table the amount of energy to deliver, and discharging the capacitor until that desired energy is reached. Alternately, the second pulse energy could be determined using the patient-dependent parameter determined in the first pulse.

The preferred embodiment as described herein applies to a biphasic waveform. The invention, however, can apply to a monophasic waveform or to multiphasic waveforms, such as triphasic, quadraphasic, etc.

While preferred embodiments of the present invention are shown and described, it is envisioned that those skilled in the art may devise various modifications of the present invention without departing from the spirit and scope of the appended claims.

I claim:

1. A method of defibrillating a patient with energy, comprising
selecting a predetermined target energy,
applying a first pulse of energy,
measuring voltage and current of said first pulse to determine a resistance of said patient,
selecting a predetermined first-pulse energy and a predetermined second-pulse energy,
truncating said first pulse after delivery of said first-pulse energy,
applying a second pulse,
truncating said second pulse after delivery of said second-pulse energy.

2. The method of claim 1, further comprising selecting said predetermined first-pulse energy and said predetermined second-pulse energy from a look-up table.

3. The method of claim 2, wherein said predetermined first-pulse energy and said predetermined second pulse energy are based on predetermined rules.

4. The method of claim 2, wherein said look-up table is generated by rules.

5. The method of claim 2, wherein said look-up table is stored in memory.

6. The method of claim 3 wherein said rules can be modified.

7. A method of defibrillating a patient with energy, comprising
selecting a predetermined target energy,
applying a first pulse of energy,
measuring voltage and current of said first pulse to determine a first resistance of said patient,
selecting a predetermined first-pulse energy,
truncating said first pulse after delivery of said first-pulse energy,
applying a second pulse,
measuring voltage and current of said second pulse to determine a second resistance of said patient,
selecting a predetermined second-pulse energy,
truncating said second pulse after delivery of said second-pulse energy.

8. The method of claim 7, further comprising selecting said predetermined first-pulse energy and said predetermined second-pulse energy from a look-up table.

9. The method of claim 7, wherein said predetermined first-pulse energy and said predetermined second pulse energy are based on predetermined rules.

10. The method of claim 8, wherein said look-up table is generated by rules.

11. The method of claim 8, wherein said look-up table is stored in memory.

12. The method of claim 10 wherein said rules can be modified.

13. A method of defibrillating a patient with energy, comprising
selecting a predetermined target energy,
applying a first pulse of energy,
determining at least one patient-dependent parameter,
selecting a predetermined first-pulse energy and a predetermined second-pulse energy,
truncating said first pulse after delivery of said first-pulse energy,
applying a second pulse,
truncating said second pulse after delivery of said second-pulse energy.

14. The method of claim 13, further comprising selecting said predetermined first-pulse energy and said predetermined second-pulse energy from a look-up table.

15. The method of claim 13, wherein said predetermined first-pulse energy and said predetermined second pulse energy are based on predetermined rules.

16. The method of claim 14, wherein said look-up table is generated by rules.

17. The method of claim 14, wherein said look-up table is stored in memory.

18. The method of claim 15 wherein said rules can be modified.

19. A method of defibrillating a patient with energy, comprising
selecting a predetermined target energy,
applying a first pulse of energy,
determining at least one patient-dependent parameter during said first pulse of energy,
selecting a predetermined first-pulse energy,
truncating said first pulse after delivery of said first-pulse energy,
applying a second pulse,
determining at least one patient-dependent parameter during said second pulse of energy,
selecting a predetermined second-pulse energy,
truncating said second pulse after delivery of said second-pulse energy.

20. The method of claim 19, further comprising selecting said predetermined first-pulse energy and said predetermined second-pulse energy from a look-up table.

21. The method of claim 19, wherein said predetermined first-pulse energy and said predetermined second pulse energy are based on predetermined rules.

22. The method of claim 20, wherein said look-up table is generated by rules.

23. The method of claim 20, wherein said look-up table is stored in memory.

24. The method of claim 21, wherein said rules can be modified.

25. A method of defibrillating a patient by delivery of at least one energy pulse, comprising
selecting a predetermined target energy
applying said at least one energy pulse to said patient
measuring voltage and current of said at least one energy pulse to determine a resistance of said patient,
selecting a predetermined value of energy,
truncating said delivery of said at least one energy pulse after delivery of said predetermined value of energy.

26. The method of claim 25, further comprising selecting said predetermined value of energy from a look-up table.

27. The method of claim 25, wherein said predetermined value of energy is based on predetermined rules.

28. The method of claim 26, wherein said look-up table is generated by rules.

29. The method of claim 26, wherein said look-up table is stored in memory.

30. The method of claim 27 wherein said rules can be modified.

31. An apparatus for delivery of at least one energy pulse to a patient, said at least one energy pulse having characteristics, wherein said characteristics are determined by an expert system.

32. The apparatus of claim 31, wherein said apparatus further comprises means for determining at least one patient-dependent parameter.

33. The apparatus of claim 32, wherein said apparatus records said characteristics, the determined at least one patient-dependent parameter, and the efficacy of said delivery of said at least one energy pulse.

34. The apparatus of claim 33, further comprising means for modifying said characteristics based on said recorded efficacy.

35. The apparatus of claim 33, further comprising means for modifying said characteristics based on said determined patient-dependent parameters.

36. The apparatus of claim 34, wherein said means is comprised of a look-up table, a neural network, a fuzzy-logic based system, genetic algorithm, adaptive performance measures, or error surface searching.

37. The apparatus of claim 35, wherein said means is comprised of a look-up table, a neural network, a fuzzy-logic based system, genetic algorithm, adaptive performance measures, or error surface searching.

38. A method of defibrillating a patient by delivery of at least one energy pulse, comprising
selecting a predetermined target energy
applying said at least one energy pulse to said patient
determining at least one patient-dependent parameter,
selecting a predetermined value of energy,
truncating said delivery of said at least one energy pulse after delivery of said predetermined value of energy.

39. The method of claim 38, further comprising selecting said predetermined value of energy from a look-up table.

40. The method of claim 38, wherein said predetermined value of energy is based on predetermined rules.

41. The method of claim 39, wherein said look-up table is generated by rules.

42. The method of claim 39, wherein said look-up table is stored in memory.

43. The method of claim 40, wherein said rules can be modified.
